(19) United States
(12) Patent Application Publication
(10) Pub. No.: US 2018/0283930 A1
(43) Pub. Date: Oct. 4, 2018

(54) WEIGHT SCALE DEVICE AND ANIMAL TOILET

(71) Applicant: SHARP KABUSHIKI KAISHA, Sakai City, Osaka (JP)

(72) Inventors: HIROKAZU ISHIKAWA, Sakai City (JP); KATSUNORI IKEDA, Sakai City (JP); AYAKO KUGA, Sakai City (JP); MASAYUKI HIRATA, Sakai City (JP); SHUJI DAIOKU, Sakai City (JP)

(21) Appl. No.: 15/765,835

(22) PCT Filed: Sep. 27, 2016
(86) PCT No.: PCT/JP2016/078520

§ 371 Date: Apr. 4, 2018

(30) Foreign Application Priority Data
Dec. 18, 2015 (JP) 2015-248055

(51) Int. Cl. G01G 17/08; A01K 1/01

(52) U.S. Cl. CPC G01G 17/08; A01K 1/0107

(57) ABSTRACT

A weight measuring device which allows an accurate measurement of a weight of a pet is realized. A pet toilet (1) includes a first weight scale (21) configured to measure a weight of an excretion of an animal and a second weight scale (22) configured to measure a weight of the animal.

### TECHNICAL FIELD

The present invention relates to a weight measuring device and an animal toilet.

### BACKGROUND ART

Patent Literature 1 discloses an automatic weight measuring system for pets, which includes a weight measuring means and a weight calculating means. The weight measuring means, provided underneath a table for pets, measures a weight of the table in (i) a state where a pet is on the table and (ii) a state where no pet is on the table. The weight calculating means calculates, based on an extent to which weight data of the table outputted from the weight measuring means has changed, a weight of the pet, and provides the weight thus calculated via a display output.

### CITATION LIST

### Patent Literature

[Patent Literature 1]

Japanese Patent Application Publication Tokukai No. 2007-330200 (Publication date: Dec. 27, 2007)

### SUMMARY OF INVENTION

### Technical Problem

The automatic weight measuring system for pets disclosed in Patent Literature 1 sometimes fails to make an accurate measurement of a weight of a pet or a weight of an excretion. For example, in a case where a pet steps onto the table with an object and then steps off the table with the object left on the table, the automatic weight measuring system for pets cannot distinguish between a weight of the object and a weight of an excretion, from an amount of change in weight. The automatic weight measuring system for pets may therefore make an erroneous measurement.

The present invention was attained in view of the above problem, and an object of the present invention is to provide a weight measuring device which allows an accurate measurement of a weight of a pet.

### Solution to Problem

A weight measuring device in accordance with an aspect of the present invention includes: a first weight scale configured to measure a weight of an excretion of an animal; and a second weight scale configured to measure (i) a weight of the animal or (ii) a total weight of the animal and the excretion.

### Advantageous Effects of Invention

An aspect of the present invention makes it possible to accurately specify a weight of an animal.

### BRIEF DESCRIPTION OF DRAWINGS

FIG. 1 is a view showing a configuration of a pet toilet in accordance with Embodiment 1 of the present invention.

FIG. 2 is a sectional view of the pet toilet.

FIG. 3 is a perspective view of the pet toilet.

FIG. 4 is an exploded perspective view of the pet toilet.

FIG. 5 is a block diagram showing a configuration of a control device.

FIG. 6 is a view showing a configuration of a pet toilet in accordance with Embodiment 2 of the present invention.

FIG. 7 is a sectional view of the pet toilet.

### DESCRIPTION OF EMBODIMENTS

### Embodiment 1

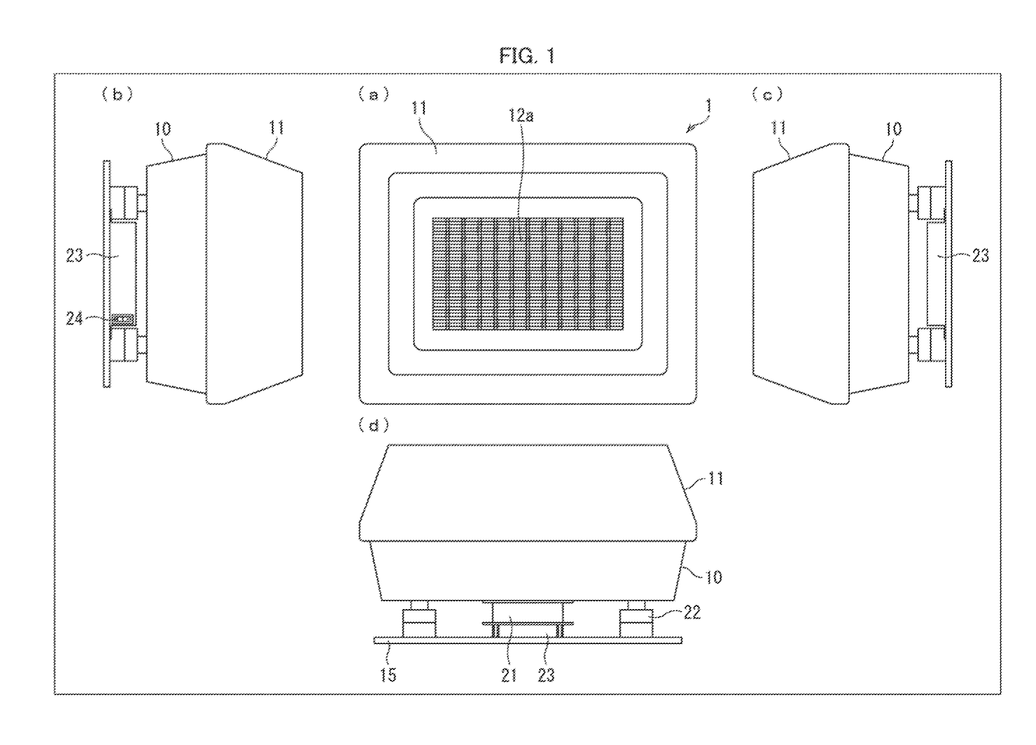

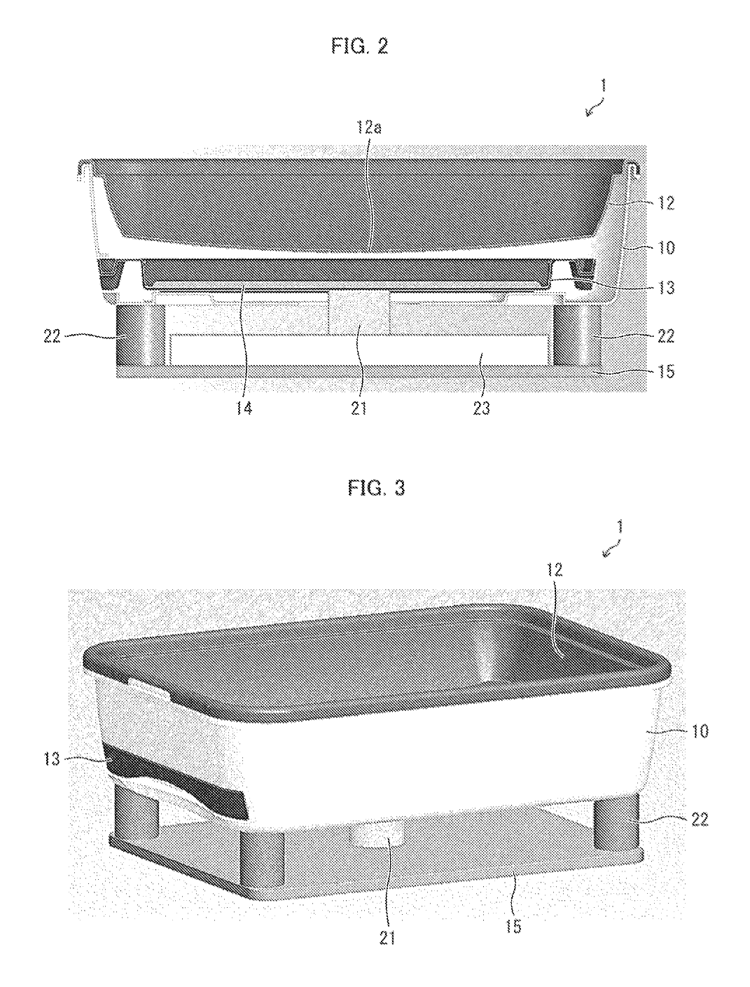

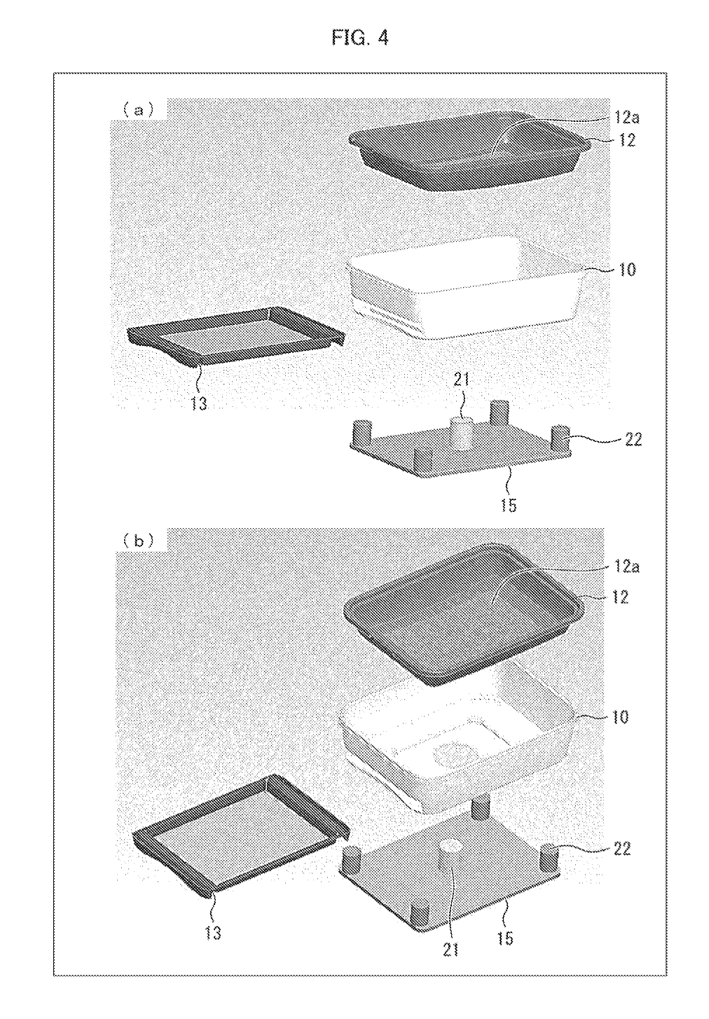

FIG. 1 is a view showing a configuration of a pet toilet in accordance with Embodiment 1. (a) of FIG. 1 is a plan view of a pet toilet 1, (b) and (c) of FIG. 1 are each a lateral view of the pet toilet 1, and (d) of FIG. 1 is a front view of the pet toilet 1. FIG. 2 is a sectional view of the pet toilet 1. FIG. 3 is a perspective view of the pet toilet 1. (a) and (b) of FIG. 4 are each an exploded perspective view of the pet toilet 1. The drawings each schematically show the configuration of the pet toilet 1.

The pet toilet 1 (animal toilet) serves as a weight measuring device which measures a weight of an animal (i.e., pet). The pet toilet 1 includes a main container 10, a cover 11, a measurement table 12, an excretion tray 13 (excretion receiver), an absorbing sheet 14 (urine pad), a supporting part 15, a first weight scale 21, second weight scales 22, and a control device 23. Note that the first weight scale 21 is omitted in each of (b) and (c) of FIG. 1. The cover 11 is omitted in each of FIGS. 2 through 4. The control device 23 is omitted in each of FIGS. 3 and 4.

The main container 10 supports the cover 11 and the measurement table 12. The main container 10 can also support a part of the excretion tray 13. The main container 10 has a hole which is formed in a central region (i.e., a region corresponding to the first weight scale 21) of a bottom part thereof. The cover 11 covers three lateral sides of the animal on the pet toilet 1.

The measurement table 12 is a table onto which the animal steps and excretes. The measurement table 12 has a mesh 12a formed in its bottom surface. The mesh 12a allows an excretion (i.e., urine) of the animal to pass therethrough. The excretion then falls onto the excretion tray 13. The mesh 12a in accordance with Embodiment 1 allows a liquid to pass therethrough, but not feces, an object (e.g., toy) in the animal's mouth, and the like. Note that, instead of the mesh 12a, the measurement table 12 can alternatively have a hole which allows the excretion to pass therethrough. The measurement table 12 in accordance with Embodiment 1 is shaped like a recessed container. However, the measurement table 12 can be arbitrarily shaped, provided that the animal can be placed on the measurement table 12 for a weight measurement.

The excretion tray 13, provided beneath the measurement table 12, is a member which receives the excretion. An area, in which the excretion tray 13 can receive the excretion, includes a region in which the mesh 12a of the measurement table 12 is formed. An absorbing sheet 14 can be set on the excretion tray 13. The absorbing sheet 14 is a sheet which absorbs a liquid such as urine. The excretion tray 13 can be attached to and detached from the main container 10 via a hole formed in a side surface of the main container 10.

The supporting part 15 is a stand plate which supports the first weight scale 21 and the second weight scales 22. The control device 23 in accordance with Embodiment 1 is provided on the supporting part 15, and the first weight scale 21 is provided on the control device 23. The supporting part 15 can thus support the first weight scale 21 via the control device 23.

The first weight scale 21 is in contact with the excretion tray 13 via the hole formed in the bottom part of the main container 10, so as to support the excretion tray 13. Examples of the first weight scale 21 include a load cell. The first weight scale 21 measures a weight of the excretion tray 13 including respective weights of the absorbing sheet 14 and the excretion. Respective Weights of the measurement table 12 and the animal are not exerted on the excretion tray 13. The first weight scale 21 supplies a measured weight to the control device 23. Note that the excretion tray 13 is so soft that the excretion tray 13 can bend due to the excretion of the weight, even in a case where the excretion tray 13 is partially supported by the main container 10. The first weight scale 21 can therefore measure a change in weight of the excretion tray 13, which change is caused by presence and absence of the excretion.

Note that, although a single first weight scale 21 is provided in Embodiment 1, a plurality of first weight scales 21 can alternatively be provided for measuring the weight of the excretion tray 13. Note that a weight to be measured by the first weight scale 21 is smaller than that to be measured by the second weight scales 22. The first weight scale 21 can therefore be smaller in maximum measurable load than the second weight scales 22. This allows (i) simplification of a structure of the first weight scale 21 and (ii) downsizing of the first weight scale 21. Furthermore, the first weight scale 21 can be higher in measuring accuracy than the second weight scales 22. This allows (i) a reduction in price of the pet toilet 1 and (ii) an improvement in measuring accuracy.

The second weight scales 22 support a structure which includes the measurement table 12. The second weight scales 22 in accordance with Embodiment 1 support a structure which includes the measurement table 12, the main container 10, and the cover 11. The four second weight scales 22 are provided so as to be in contact with respective four corners of the bottom part of the main container 10. Examples of the second weight scales 22 include a load cell. The second weight scales 22 measure a total weight of (i) the structure including the measurement table 12, the main container 10, and the cover 11 and (ii) the animal. A weight of the excretion is not (or almost not) exerted on the second weight scales 22. The second weight scales 22 supplies a measured weight to the control device 23.

Based on respective weights measured by the first weight scale 21 and the second weight scales 22, the control device 23 calculates the respective weights of the animal and the excretion. The control device 23 in accordance with Embodiment 1 includes a housing which houses therein components such as a control substrate. The control device 23 includes a power switch 24.

(Configuration of Control Device)

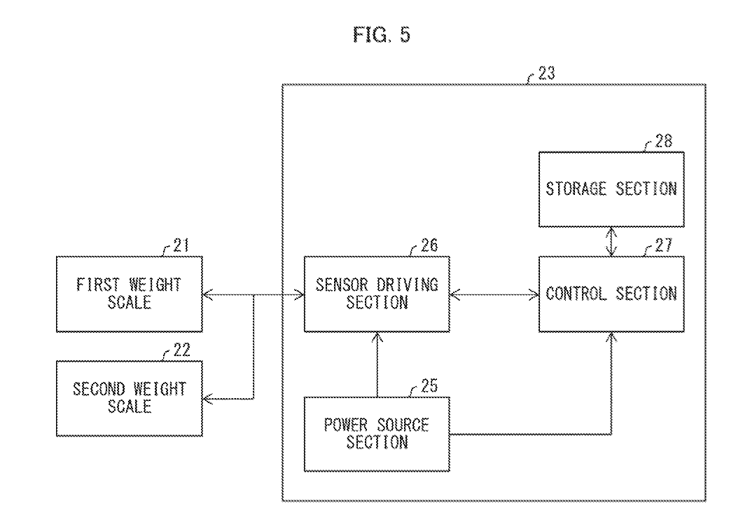

FIG. 5 is a block diagram showing a configuration of the control device 23. The control device 23 includes a power source section 25, a sensor driving section 26, a control section 27, and a storage section 28. Respective weights, which are measured by the first weight scale 21 and the second weight scales 22, are inputted to the sensor driving section 26.

The power source section 25 supplies electric power to each section of the control device 23 (i.e., to each of the sensor driving section 26, the control section 27, and the storage section 28). The power switch 24 turns ON or OFF the electric power which is to be supplied from the power source section 25 to the each section. The power source section 25 can include, for example, a battery.

The sensor driving section 26 supplies electric power to the first weight scale 21 and the second weight scales 22 so that the first weight scale 21 and the second weight scales 22 operate. The sensor driving section 26 also receives respective measured weights from the first weight scale 21 and the second weight scales 22. The sensor driving section 26 causes the first weight scale 21 and the second weight scales 22 to make measurements at respective given timings (e.g., at certain cycle). The sensor driving section 26 supplies the measured weights thus received to the control section 27.

The control section 27 calculates the respective weights of the animal and the excretion. The weight measured by the first weight scale 21 indicates the weight of the excretion tray 13 which includes respective weights of the absorbing sheet 14 and the excretion. The control section 27 determines, as a time point at which the animal excreted, a time point at which the weight measured by the first weight scale 21 changed (i.e., a time point at which a measured weight became heavy). Based on an amount by which the measured weight changed, the control section 27 specifies the weight of the excretion. More specifically, the control section 27 specifies the weight of the excretion by subtracting, from a weight measured after the excretion, a weight measured before the excretion.

The weight measured by the second weight scales 22 indicates the total weight of (i) the structure including the measurement table 12, the main container 10, and the cover 11 and (ii) the animal. In a case where no animal is on the measurement table 12, a weight derived from the animal is regarded as 0. The control section 27 determines, as a time point at which the animal stepped onto the measurement table 12, a time point at which the weight measured by the second weight scales 22 changed (i.e., a time point at which a measured weight became heavy). In a case where the animal excretes after stepping onto the measurement table 12, the weight measured by the second weight scales 22 decreases by just that much, whereas the weight measured by the first weight scale 21 increases by just that much. The control section 27 determines, as a time point at which the animal stepped off the measurement table 12, a time point at which the weight measured by the second weight scales 22 decreased out of a period of time during which there is no change in weight measured by the first weight scale 21. Note that a period of time, during which data (i.e., measured weight) changes within a given range, can be broadly regarded as “a period of time during which there is no change in measured weight.”

The control section 27 determines that (i) the animal has left an object on the measurement table 12 or (ii) the animal has gone away with an object from the measurement table 12, in a case where (a) a weight, which is measured by the second weight scales 22 before the animal steps onto the measurement table 12, differs from (b) a weight which is measured by the second weight scales 22 after the animal steps off the measurement table 12. The control section 27 specifies, as a weight of the object, a difference between (i) the weight which is measured by the second weight scales 22 before the animal steps onto the measurement table 12 and (ii) the weight which is measured by the second weight scales 22 after the animal steps off the measurement table 12. In a case where the weight measured after the animal steps off is larger, it means that the animal has left the object on the measurement table 12. In a case where the weight measured before the animal steps onto the measurement table 12 is larger, the animal has gone away with an object from the measurement table 12. The control section 27 treats the weight of the object as 0, in a case where (i) the weight, which is measured by the second weight scales 22 before the animal steps onto the measurement table 12, is equal to (ii) the weight which is measured by the second weight scales 22 after the animal steps off the measurement table 12.

The control section 27 specifies the weight of the animal by subtracting the following weights (i) and (ii) from the following weight (iii). The weight (i) indicates a weight which is measured by the second weight scales 22 after the animal steps off the measurement table 12. The weight (ii) indicates the weight of the object. The weight (iii) indicates a weight which is measured by the second weight scales 22 during a period of time between (a) a time point at which the animal excreted and (b) a time point at which the animal stepped off the measurement table 12. That is, the control section 27 specifies the weight of the animal based on a change in weight. Alternatively, the control section 27 specifies the weight of the animal by subtracting the following weights (i) and (ii) from the following weight (iii). The weight (i) indicates a weight which is measured by the second weight scales 22 before the animal steps onto the measurement table 12. The weight (ii) indicates the weight of the object. The weight (iii) indicates a weight which is measured by the second weight scales 22 during a period of time between (a) a time point at which the animal stepped onto the measurement table 12 and (b) a time point at which the animal excreted. This allows the control section 27 to accurately specify the weight of an animal, without being affected by the respective weights of the object and the excretion.

The control section 27 controls the storage section 28 to store therein the respective weights of the animal and the excretion. Note that the control section 27 can be configured to send the respective weights of the animal and the excretion to an external device. The storage section 28 can be a detachable storage medium. Moreover, the pet toilet 1 can include a display device, and the control section 27 can control the display device to display the weight of the animal and/or the weight of the excretion. The control section 27 can also control the storage section 28 to store therein time at which the animal excreted. A user can accurately grasp behavior of the animal, by (i) causing another computer to read out data stored in the storage section 28 or (ii) observing the data via the display device. The user can learn, for example, the number of times (i.e., how often) the animal excretes after stepping onto the pet toilet 1 and the number of times (i.e., how often) the animal does not excrete after stepping onto the pet toilet 1.

### Embodiment 21

The following description will discuss Embodiment 2 of the present invention. For convenience, identical reference numerals are given to members having respective functions identical to those of the members described in Embodiment 1, and descriptions of such members are omitted in Embodiment 2.

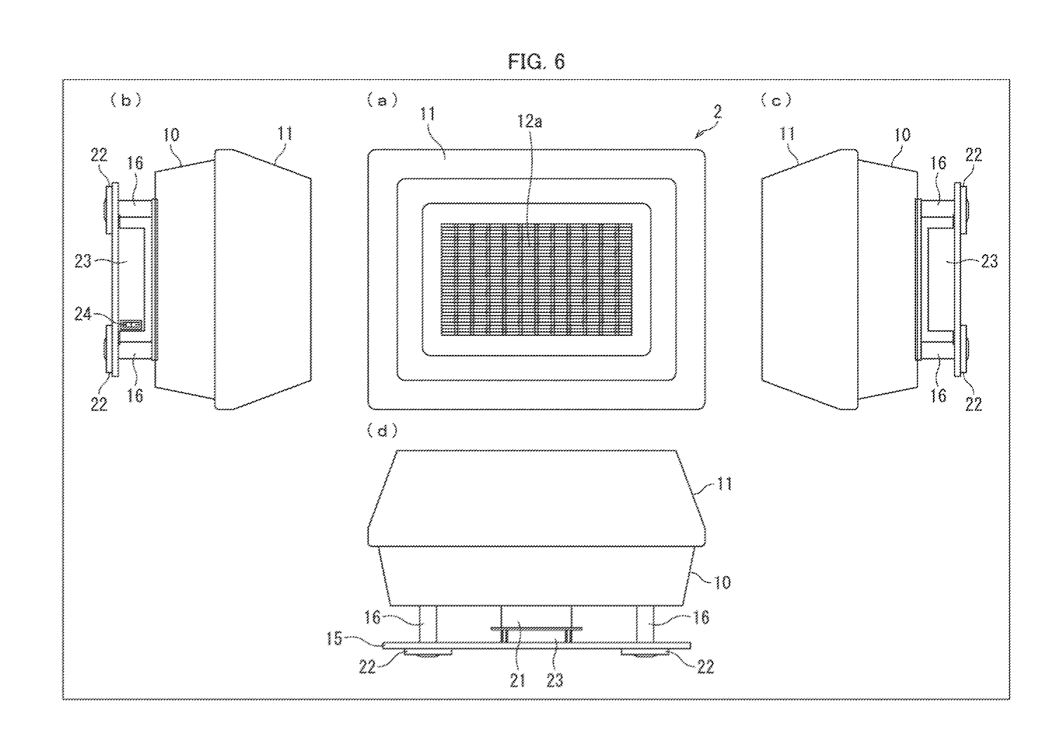

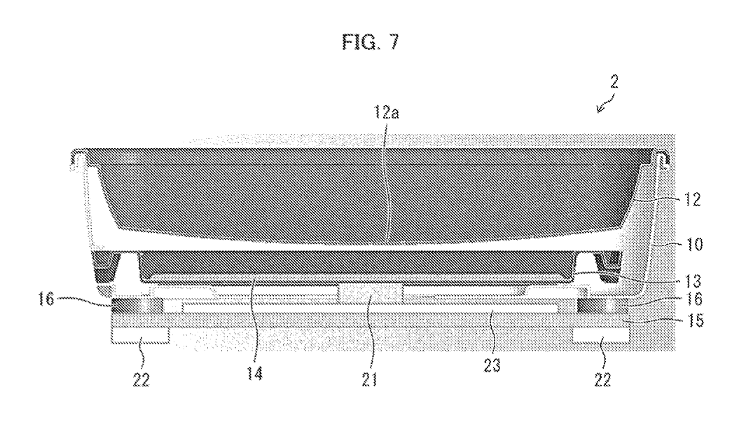

FIG. 6 is a view showing a configuration of a pet toilet in accordance with Embodiment 2. (a) of FIG. 6 is a plan view of a pet toilet 2, (b) and (c) of FIG. 6 are each a lateral view of the pet toilet 2, and (d) of FIG. 6 is a front view of the pet toilet 2. FIG. 7 is a sectional view of the pet toilet 2. A first weight scale 21 is omitted in each of (b) and (c) of FIG. 6. A cover 11 is omitted in FIG. 7. The drawings each schematically show the configuration of the pet toilet 2.

The pet toilet 2 serves as a weight measuring device which measures a weight of an animal (i.e., pet). The pet toilet 2 includes a main container 10, the cover 11, a measurement table 12, an excretion tray 13, an absorbing sheet 14, a supporting part 15, posts 16 (second supporting parts), the first weight scale 21, second weight scales 22, and a control device 23.

In Embodiment 2, the four second weight scales 22 are provided beneath the supporting part 15. Lower ends of the respective second weight scales 22 are in contact with, for example, a floor so that the second weight scales 22 support the supporting part 15.

The supporting part 15 is a stand plate which supports the first weight scale 21 and the main container 10. The control device 23 in accordance with Embodiment 2 is provided on the supporting part 15, and the first weight scale 21 is provided on the control device 23. The four posts 16 are provided on the supporting part 15 so as to support the main container 10. Alternatively, the supporting part 15 can directly support the main container 10, instead of the posts 16 being provided.

The first weight scale 21 measures a weight of the excretion tray 13 including respective weights of the absorbing sheet 14 and an excretion. Respective weights of the measurement table 12 and the animal are not exerted on the excretion tray 13. The first weight scale 21 supplies a measured weight to the control device 23.

The second weight scales 22 measure a weight of a structure which includes respective weights of the measurement table 12, the main container 10, the cover 11, the supporting part 15, the control section 27, the first weight scale 21, and the excretion tray 13. The weight of the excretion tray 13 includes respective weights of the absorbing sheet 14 and the excretion. The second weight scales 22 supplies a measured weight to the control device 23.

The control device 23 includes, as with the case of FIG. 5 in Embodiment 1, a power source section 25, a sensor driving section 26, a control section 27, and a storage section 28.

The control section 27 calculates the respective weights of the animal and the excretion. The weight measured by the first weight scale 21 indicates the weight of the excretion tray 13 including respective weights of the absorbing sheet 14 and the excretion. The control section 27 determines, as a time point at which the animal excreted, a time point at which the weight measured by the first weight scale 21 changed (i.e., a time point at which a measured weight became large). The control section 27 specifies the weight of the excretion by subtracting, from a weight measured after the excretion, a weight measured before the excretion.

The weight measured by the second weight scales 22 in accordance with Embodiment 2 indicates a total weight of (i) a weight of the structure including respective weights of the measurement table 12, the main container 10, the cover 11, the supporting part 15, the control section 27, the first weight scale 21, and the excretion tray 13, (ii) a weight of the animal, and (iii) a weight of the excretion. In a case where no animal is on the measurement table 12, a weight derived from the animal is regarded as 0. The control section 27 determines, as a time point at which the animal stepped onto the measurement table 12, a time point at which the weight measured by the second weight scales 22 changed (i.e., a time point at which a measured weight became heavy). Note that no change occurs in weight measured by the second weight scales 22 even if the animal excretes after stepping onto the measurement table 12. The control section 27 determines, as a time point at which the animal stepped off the measurement table 12, a time point at which the weight measured by the second weight scales 22 decreased.

The control section 27 determines that (i) the animal has left an object on the measurement table 12 or (ii) the animal has gone away with an object from the measurement table 12, in a case where (a) a weight, which is measured by the second weight scales 22 before the animal steps onto the measurement table 12, differs from (ii) a weight which is obtained by subtracting the weight of the excretion from a weight which is measured by the second weight scales 22 after the animal steps off the measurement table 12. The control section 27 specifies, as a weight of the object, a difference between (i) the weight which is measured by the second weight scales 22 before the animal steps onto the measurement table 12 and (ii) the weight which is obtained by subtracting the weight of the excretion from the weight which is measured by the second weight scales 22 after the animal steps off the measurement table 12. In a case where the weight obtained by subtracting the weight of the excretion from the weight measured after the animal steps off is larger, it means that the animal has left an object on the measurement table 12. In a case where the weight measured before the animal steps on is larger, it means that the animal has gone away with an object from the measurement table 12. The control section 27 treats the weight of the object as 0, in a case where (i) the weight which is measured by the second weight scales 22 before the animal steps onto the measurement table 12 is equal to (ii) the weight which is obtained by subtracting the weight of the excretion from the weight which is measured by the second weight scales 22 after the animal steps off the measurement table 12.

The control section 27 specifies the weight of the animal by subtracting the following weights (i) and (ii) from the following weight (iii). The weight (i) indicates a weight which is measured by the second weight scales 22 after the animal steps off the measurement table 12. The weight (ii) indicates the weight of the object. The weight (iii) indicates a weight which is measured by the second weight scales 22 during a period of time between (a) a time point at which the animal stepped onto the measurement table 12 and (b) a time point at which the animal stepped off the measurement table 12. Alternatively, the control section 27 specifies the weight of the animal by subtracting the following weights (i) through (iii) from the following weight (iv). The weight (i) indicates a weight which is measured by the second weight scales 22 before the animal steps onto the measurement table 12. The weight (ii) indicates the weight of the excretion. The weight (iii) indicates the weight of the object. The weight (iv) indicates the weight which is measured by the second weight scales 22 during a period of time between (a) a time point at which the animal stepped onto the measurement table 12 and (b) a time point at which the animal stepped off the measurement table 12. This allows the control section 27 to accurately specify the weight of the animal, without being affected by the respective weights of the object and the excretion.

Note that, although each of Embodiments 1 and 2 has discussed the example where the absorbing sheet 14 is provided on the excretion tray 13, the absorbing sheet 14 can be omitted. Even in a case where no absorbing sheet 14 is provided, no problem arises in a weight measuring function. This is because the excretion of the animal stays in the excretion tray 13.

Each of Embodiments 1 and 2 has discussed a case where the four second weight scales 22 are provided beneath the main container 10, but the number of the second weight scales 22 is not limited as such. Alternatively, the second weight scales 22 can be a single scale or a plurality of (not four) scales that are provided in a scattered manner. For example, in Embodiment 1, in a case where a single scale, which serves as a second weight scale 22, is provided (i) near a center of the main container 10 and (ii) beneath the main container 10, it is possible to prevent the second weight scale 22 from overlapping with the first weight scale 21, by forming, in a torus-shape, a part where the second weight scale 22 receives a weight.

The above configuration also applies to the first weight scale 21. Although a single first weight scale is provided in each of Embodiments 1 and 2, the first weight scale 21 can alternatively be composed of a plurality of scales.

### Embodiment 3

### Software Implementation Example

A control block (especially each of the sensor driving section 26 and the control section 27) of the control device 23 can be realized by a logic circuit (hardware) provided in an integrated circuit (IC chip) or the like or can be alternatively realized by software as executed by a central processing unit (CPU).

In the latter case, the control device 23 includes a CPU that executes instructions of a program that is software realizing the foregoing functions; a read only memory (ROM) or a storage device (each referred to as “storage medium”) in which the program and various kinds of data are stored so as to be readable by a computer (or a CPU); and a random access memory (RAM) in which the program is loaded. An object of the present invention can be achieved by a computer (or a CPU) reading and executing the program stored in the storage medium. Examples of the storage medium encompass “a non-transitory tangible medium” such as a tape, a disk, a card, a semiconductor memory, and a programmable logic circuit. The program can be supplied to the computer via any transmission medium (such as a communication network or a broadcast wave) which allows the program to be transmitted. Note that the present invention can also be achieved in the form of a computer data signal in which the program is embodied via electronic transmission and which is embedded in a carrier wave.

[Recap]

A weight measuring device in accordance with a first aspect of the present invention includes: a first weight scale configured to measure a weight of an excretion of an animal; and a second weight scale configured to measure (i) a weight of the animal or (ii) a total weight of the animal and the excretion.

With the above configuration, it is possible to accurately specify the weight of the animal without being affected by respective weights of an object and an excretion.

The weight measuring device in accordance with a second aspect of the present invention can be configured to further include, in the first aspect of the present invention, a measurement table on which the animal is to be placed; and an excretion receiver which receives the excretion from the animal placed on the measurement table, the first weight scale measuring a change in weight of the excretion receiver, the second weight scale measuring a change in weight of a structure which includes the measurement table.

The weight measuring device in accordance with a third aspect of the present invention can be configured such that, in the second aspect of the present invention, the second weight scale measures the total weight of the animal and the excretion, by measuring the change in weight of the structure which includes respective weights of the measurement table and the excretion receiver.

The weight measuring device in accordance with a fourth aspect of the present invention can be configured such that, in the second or third aspect of the present invention, the measurement table has a mesh or a hole each of which passes the excretion therethrough.

The weight measuring device in accordance with a fifth aspect of the present invention can be configured such that, in the fourth aspect of the present invention, the hole or the mesh is provided so as to cause urine to pass therethrough; and the excretion receiver receives the urine.

The weight measuring device in accordance with a sixth aspect of the present invention can be configured such that, in the second through fifth aspects of the present invention, the first weight scale is provided so as to be in contact with the excretion receiver; and the second weight scale is provided so as to be in contact with the structure which includes the measurement table.

The weight measuring device in accordance with a seventh aspect of the present invention can be configured such that, in the third aspect of the present invention, the second weight scale measures the change in weight of the structure which includes a weight of the first weight scale.

The weight measuring device in accordance with an eighth aspect of the present invention can be configured such that, in any one of the first through seventh aspects of the present invention, the first weight scale is smaller in maximum measurable load and higher in measuring accuracy than the second weight scale.

An animal toilet in accordance with a ninth aspect of the present invention can include a weight measuring device in accordance with any one of the first through eighth aspects of the present invention.

The present invention is not limited to the embodiments, but can be altered by a skilled person in the art within the scope of the claims. The present invention also encompasses, in its technical scope, any embodiment derived by combining technical means disclosed in differing embodiments. Further, it is possible to form a new technical feature by combining the technical means disclosed in the respective embodiments.

### REFERENCE SIGNS LIST

1, 2: Pet toilet (weight measuring device, animal toilet)
10: Main container
11: Cover
12: Measurement table
12a: Mesh
13: Excretion tray (excretion receiver)
14: Absorbing sheet
15: Supporting part
21: First weight scale
22: Second weight scale
23: Control device
25: Power source section
26: Sensor driving section
27: Control section
28: Storage section

1. A weight measuring device, comprising:
a first weight scale configured to measure a weight of an excretion of an animal; and
a second weight scale configured to measure (i) a weight of the animal or (ii) a total weight of the animal and the excretion.

2. A weight measuring device as set forth in claim 1, further comprising:
a measurement table on which the animal is to be placed; and
an excretion receiver which receives the excretion from the animal placed on the measurement table,
the first weight scale measuring a change in weight of the excretion receiver,
the second weight scale measuring a change in weight of a structure which includes the measurement table.

3. The weight measuring device as set forth in claim 2, wherein:
the second weight scale measures the total weight of the animal and the excretion, by measuring the change in weight of the structure which includes respective weights of the measurement table and the excretion receiver.

4. The weight measuring device as set forth in claim 2, wherein:
the measurement table has a mesh or a hole each of which passes the excretion therethrough.

5. The weight measuring device as set forth in claim 4, wherein:
the hole or the mesh is provided so as to cause urine to pass therethrough; and
the excretion receiver receives the urine.

6. The weight measuring device as set forth in claim 2, wherein:
the first weight scale is provided so as to be in contact with the excretion receiver; and
the second weight scale is provided so as to be in contact with the structure which includes the measurement table.

7. The weight measuring device as set forth in claim 3, wherein:
the second weight scale measures the change in weight of the structure which includes a weight of the first weight scale.

8. The weight measuring device as set forth in claim 1, wherein
the first weight scale is smaller in maximum measurable load and higher in measuring accuracy than the second weight scale.

9. An animal toilet comprising a weight measuring device as set forth in claim 1.
